(19) United States
(12) Patent Application Publication
(10) Pub. No.: US 2006/0063714 A1
(43) Pub. Date: Mar. 23, 2006

(54) Liquid, aqueous, pharmaceutical compositions of factor VII polypeptides

(75) Inventors: Michael Bech Jensen, Allerod (DK); Birthe Lykkegaard Hansen, Vaerlose (DK); Troels Kornfelt, Virum (DK); Kirsten Kramer Jakobsen, Farum (DK); Janus Krarup, Gentofte (DK); Egon Persson, Akarp (SE); Anders Klarskov Petersen, Naerum (DK); Andrew Neil Bowler, Gentofte (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/229,427

(22) Filed: Sep. 15, 2005

Related U.S. Application Data
(63) Continuation of application No. PCT/DK04/00181, filed on Mar. 18, 2004.

(30) Foreign Application Priority Data
Mar. 18, 2003 (DK) PA 2003/00413
May 23, 2003 (DK) PA 2003/00788
Jun. 25, 2003 (DK) PA 2003/00959
Jul. 1, 2003 (DK) PA 2003/00995
Aug. 14, 2003 (DK) PA 2003/01161

(51) Int. Cl. A61K 38/36

(52) U.S. Cl. 514/12

(57) ABSTRACT

The invention relates to a liquid, aqueous pharmaceutical composition comprising a Factor VII polypeptide (e.g. human Factor VIIa) and a buffering agent; wherein the molar ratio of non-complexed calcium ions (Ca²⁺) to the Factor VII polypeptide is lower than 0.5. The composition may further comprise a stabilizing agent (e.g. copper or magnesium ions, benzamidine, or guanidine), a non-ionic surfactant, a tonicity modifying agent, an antioxidant and a preservative. The composition is useful for treating a Factor VII-responsive syndrome, such as bleeding disorders, including those caused by clotting Factor deficiencies (e.g. haemophilia A, haemophilia B, coagulation Factor XI deficiency, coagulation Factor VII deficiency); by thrombocytopenia or von Willebrand's disease, or by clotting Factor inhibitors, and intra cerebral haemorrhage, or excessive bleeding from any cause. The preparations may also be administered to patients in association with surgery or other trauma or to patients receiving anticoagulant therapy.

### CROSS-REFERENCE TO RELATED PATENT APPLICATIONS

This patent application is a continuation of PCT Patent Application No. DK2004/000181, filed Mar. 18, 2004 and claims the benefit of U.S. Provisional Patent Application Nos. 60/457,809, filed Mar. 26, 2003, U.S. 60/476,280 filed Jun. 5, 2003, U.S. 60/484,334 filed Jul. 2, 2003, U.S. 60/485,334 filed Jul. 7, 2003, and U.S. 60/496,443 filed Aug. 20, 2003, and Danish Patent Application Nos. PA 2003/00413 filed Mar. 18, 2003, PA 2003/00788 filed May 23, 2003, PA 2003/00959 filed Jun. 25, 2003, PA 2003/00995 filed Jul. 1, 2003, and PA 2003/01161 filed Aug. 14, 2003.

### FIELD OF THE INVENTION

The present invention is directed to liquid, aqueous pharmaceutical compositions containing Factor VII polypeptides, and methods for preparing and using such compositions, as well as containers containing such compositions, and the use of such compositions in the treatment of a Factor VII-responsive syndrome. More particularly, the invention relates to liquid compositions stabilised against chemical and/or physical degradation.

### BACKGROUND OF THE INVENTION

A variety of Factors involved in the blood clotting process have been identified, including Factor VII (FVII), a plasma glycoprotein. Coagulation is initiated by the formation of a complex between Tissue Factor (TF) being exposed to the circulating blood following an injury to the vessel wall, and FVIIa which is present in the circulation in an amount corresponding to about 1% of the total FVII protein mass. FVII exists in plasma mainly as a single-chain zymogen which is cleaved by FXa into its two-chain, activated form, FVIIa. Recombinant activated Factor VIIa (rFVIIa) has been developed as a pro-haemostatic agent. The administration of rFVIIa offers a rapid and highly effective pro-haemostatic response in haemophilic subjects with bleedings, who cannot be treated with other coagulation Factor products due to antibody formation. Also bleeding in subjects with Factor VII deficiency or subjects having a normal coagulation system but experiencing excessive bleeding can be treated successfully with FVIIa.

It is desirable to have administration forms of Factor VIIa suitable for both storage and for delivery. Ideally, the drug product is stored and administered as a liquid. Alternatively, the drug product is lyophilized, i.e. freeze-dried, and then reconstituted by adding a suitable diluent prior to patient use. Ideally, the drug product has sufficient stability to be kept in long-term storage, i.e. more than six months.

The decision to either maintain the finished drug product as a liquid or to freeze-dry it is usually based on the stability of the protein drug in those forms. Protein stability can be affected inter alia by such Factors as ionic strength, pH, temperature, repeated cycles of freeze/thaw, and exposures to shear forces. Active protein may be lost as a result of physical instabilities, including denaturation and aggregation (both soluble and insoluble aggregate formation), as well as chemical instabilities, including, for example, hydrolysis, deamidation, isomerization, and oxidation, to name just a few. For a general review of the stability of protein pharmaceuticals, see, for example, Manning, et al., Pharmaceutical Research 6:903-918 (1989).

While the possible occurrence of protein instabilities is widely appreciated, it is impossible to predict particular instability problems of a particular protein. Any of these instabilities can result in the formation of a protein by-product, or derivative, having lowered activity, increased toxicity, and/or increased immunogenicity. Indeed, protein precipitation may lead to thrombosis, non-homogeneity of dosage form and amount, as well as clogged syringes. Furthermore, post-translational modifications such as, for example, gamma carboxylation of certain glutamic acid residues in the N-terminus and addition of carbohydrate side chains provide potential sites that may be susceptible to modification upon storage. Also, specific to Factor VIIa, being a serine protease, fragmentation due to autocatalysis may occur (enzymatic degradation). Thus, the safety and efficacy of any composition of a protein is directly related to its stability. Maintaining stability in a liquid form is generally different from maintaining stability in a lyophilized form because of highly increased potential for molecular motion and thereby increased probability of molecular interactions. Maintaining stability in a concentrated form is also different from the above, because of the propensity for aggregate formation at increased protein concentrations.

When developing a liquid composition, many Factors are taken into consideration. Short-term, i.e. less than six months, liquid stability generally depends on avoiding gross structural changes, such as denaturation and aggregation. These processes are described in the literature for a number of proteins, and many examples of stabilizing agents exist. It is well-known that an agent effective in stabilizing one protein actually acts to destabilize another. Once the protein has been stabilized against gross structural changes, developing a liquid composition for long-term stability (e.g., greater than six months) depends on further stabilizing the protein from types of degradation specific to that protein. More specific types of degradation may include, for example, disulfide bond scrambling, oxidation of certain residues, deamidation, cyclization. Although it is not always possible to pinpoint the individual degradation species, assays are developed to monitor subtle changes so as to monitor the ability of specific excipients to uniquely stabilize the protein of interest.

It is desirable that the pH of the composition is in a physiologically suitable range upon injection/infusion, otherwise pain and discomfort for the patient may result.

For a general review of protein compositions, see, for example, Cleland et al.: The development of stable protein compositions: A closer look at protein aggregation, deamidation and oxidation, Critical Reviews in Therapeutic Drug Carrier Systems 1993, 10(4): 307-377; and Wang et al., Parenteral compositions of proteins and peptides: Stability and stabilizers, Journal of Parenteral Science and Technology 1988 (Supplement), 42 (2S).

Factor VIIa degrades via several pathways, especially aggregation (dimerisation), oxidation, and autolytic cleavage (clipping of the peptide backbone). Furthermore, precipitation may occur. Many of these reactions can be slowed significantly by removal of water from the protein. However, the development of an aqueous composition for Factor VIIa has the advantages of eliminating reconstitution errors, thereby increasing dosing accuracy, as well as simplifying the use of the product clinically, thereby increasing patient compliance. Ideally, compositions of Factor VIIa should be stable for more than 6 months over a wide range of protein concentrations. This allows-for flexibility in methods of administration. Generally, more highly concentrated forms allow for the administration of lower volumes, which is highly desirable from the patients' point of view. Liquid compositions can have many advantages over freeze-dried products with regard to ease of administration and use.

Today, the only commercially available, recombinantly-made FVII polypeptide composition is a freeze-dried Factor FVIIa product which is reconstituted before use; it contains a relatively low Factor VIIa concentration, e.g., about 0.6 mg/mL. A vial (1.2 mg) of NovoSeven® (Novo Nordisk A/S, Denmark) contains 1.2 mg recombinant human Factor VIIa, 5.84 mg NaCl, 2.94 mg CaCl₂, 2H₂O, 2.64 mg GlyGly, 0.14 mg polysorbate 80, and 60.0 mg mannitol; it is reconstituted to pH 5.5 by 2.0 mL water for injection (WFI). When reconstituted, the protein solution is stable for use for 24 hours. Thus, no liquid ready-for-use- or concentrated Factor VII products are currently commercially available.

WO 03/055512 discloses liquid, aqueous pharmaceutical composition comprising a Factor VII polypeptide, a buffer and an agent selected from a calcium salt, a magnesium salt and a mixture thereof, in particular a calcium salt, in a concentration of at least 15 mM. The calcium/magnesium salt provides stability to the liquid, aqueous composition.

In view of the above, it is an objective of this invention to provide further liquid, aqueous Factor VII polypeptide pharmaceutical compositions which provide acceptable control of chemical and/or physical degradation products such as enzymatic degradation or autocatalysis products.

### SUMMARY OF THE INVENTION

The present inventors have now found that although a number of prior art references recommend the use of a relatively high concentration of calcium ions in purification steps and in aqueous liquids for storage of Factor VII polypeptides, it is also possible to obtain excellent storage stability for liquid, aqueous pharmaceutical compositions of Factor VII polypeptides by ensuring that the relative ratio between calcium ions (Ca²⁺) and the Factor VII polypeptide is very low.

Thus, one aspect of the present invention relates to a liquid, aqueous pharmaceutical composition comprising a Factor VII polypeptide (i) and a buffering agent (ii) suitable for keeping pH in the range of from about 5.0 to about 9.0; wherein the molar ratio of non-complexed calcium ions (Ca²⁺) to the Factor VII polypeptide is lower than 0.5.

A second aspect of the present invention relates to a method for preparing a liquid, aqueous pharmaceutical composition of a Factor VII polypeptide comprising the step of providing the Factor VII polypeptide (i) in a solution comprising a buffering agent (ii) suitable for keeping pH in the range of from about 5.0 to about 9.0; while ensuring that, in the final composition, the molar ratio of non-complexed calcium ions (Ca²⁺) to the Factor VII polypeptide is lower than 0.5.

A third aspect of the present invention relates to a liquid, aqueous pharmaceutical composition as defined above for use as a medicament.

A fourth aspect of the present invention relates to the use of a liquid, aqueous pharmaceutical composition as defined above for the preparation of a medicament for treating a Factor VII-responsive syndrome.

A fifth aspect of the present invention relates to a method for treating a Factor VII-responsive syndrome, the method comprising administering to a subject in need thereof an effective amount of a liquid, aqueous pharmaceutical composition as defined above.

A sixth aspect of the present invention relates to an air-tight, at least partially filled container containing a liquid, aqueous pharmaceutical composition as defined above, and optionally an inert gas, said container comprising (i) a wall portion and (ii) one or more closure means not constituting part of said wall portion.

### DETAILED DESCRIPTION OF THE INVENTION

As mentioned above, the present invention resides in the development of a novel stabilised liquid, aqueous pharmaceutical composition comprising a Factor VII polypeptide. More specifically, the liquid, aqueous pharmaceutical composition comprises a Factor VII polypeptide (i) and a buffering agent (ii) suitable for keeping pH in the range of from about 5.0 to about 9.0; wherein the molar ratio of non-complexed calcium ions (Ca²⁺) to the Factor VII polypeptide is lower than 0.5.

When used herein, the term “the concentration of non-complexed calcium ions” is intended to mean the difference between the total concentration of calcium ions and the concentration of calcium bound to calcium chelators. In this regard, the Factor VII polypeptide is not regarded as a “calcium chelator” although calcium is expected to bind to, or become associated with, the Factor VII polypeptide under certain conditions.

Preferably, the molar ratio of non-complexed calcium ions (Ca²⁺) to the Factor VII polypeptide is lower than 0.5, e.g. in the range of 0.001-0.499, such as 0.005-0.050, or in the range of 0.000-0.499, such as in the range of 0.000-0.050, or about 0.000. In order to obtain the low relative ratio between calcium ions (Ca²⁺) and the Factor VII polypeptide, it may be necessary or desirable to add a calcium chelator in order to bind (complex) excess calcium ions. This is particularly relevant where the ratio between calcium ions and the Factor VII polypeptide in a solution from a process step preceding the formulation step exceeds the limit stated above. Examples of “calcium chelators” include EDTA, citric acid, NTA, DTPA, tartaric acid, lactic acid, malic acid, succinic acid, HIMDA, ADA and similar compounds.

Factor VII Polypeptide (i)

The biological effect of the pharmaceutical composition is mainly ascribed to the presence of the Factor VII polypeptide, although other active ingredients may be included in combination with the Factor VII polypeptide.

As used herein, the term “Factor VII polypeptide” encompasses wild-type Factor VII (i.e. a polypeptide having the amino acid sequence disclosed in U.S. Pat. No. 4,784,950), as well as variants of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIA. Typically, Factor VII is cleaved between residues 152 and 153 to yield Factor VIIa. The term “Factor VII polypeptide” also encompasses polypeptides, including variants, in which the Factor VIIA biological activity has been substantially modified or somewhat reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (i) bind to Tissue Factor (TF) and (ii) catalyze the proteolytic cleavage of Factor 1× or Factor X to produce activated Factor 1× or X (Factor IXa or Xa, respectively).

For the purposes of the invention, biological activity of Factor VII polypeptides (“Factor VII biological activity”) may be quantified by measuring the ability of a preparation to promote blood clotting, cf. Assay 4 described herein. In this assay, biological activity is expressed as the reduction in clotting time relative to a control sample and is converted to “Factor VII units” by comparison with a pooled human serum standard containing 1 unit/mL Factor VII activity. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X. (Persson et al., J. Biol. Chem. 272:19919-19924, 1997); (ii) measuring Factor X hydrolysis in an aqueous system (“In Vitro Proteolysis Assay”, see Assay 2 below); (iii) measuring the physical binding of Factor VIIa or a Factor VII-related polypeptide to TF using an instrument based on surface plasmon resonance (Persson, FEBS Letts. 413:359-363, 1997); (iv) measuring hydrolysis of a synthetic substrate by Factor VIIa and/or a Factor VII-related polypeptide (“In Vitro Hydrolysis Assay”, see Assay 1 below); or (v) measuring generation of thrombin in a TF-independent in vitro system (see Assay 3 below).

Factor VII variants having substantially the same or improved biological activity relative to wild-type Factor VIIa encompass those that exhibit at least about 25%, such as, e.g., at least about 50%, at least about 75%, or at least about 90% of the specific activity of Factor VIIa that has been produced in the same cell type, when tested in one or more of a clotting assay (Assay 4), proteolysis assay (Assay 2), or TF binding assay as described above. Factor VII variants having substantially reduced biological activity relative to wild-type Factor VIIa are those that exhibit less than about 25%, such as, e.g., less than about 10%, or less than about 5% of the specific activity of wild-type Factor VIIa that has been produced in the same cell type when tested in one or more of a clotting assay (Assay 4), proteolysis assay (Assay 2), or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189; and FVII variants exhibiting increased proteolytic stability as disclosed in WO 02/38162 (Scripps Research Institute); FVII variants having a modified Gla-domain and exhibiting an enhanced membrane binding as disclosed in WO 99/20767 (University of Minnesota); and FVII variants as disclosed in WO 01/58935 (Maxygen ApS).

Non-limiting examples of Factor VII variants having increased biological activity compared to wild-type FVIIa include FVII variants as disclosed in WO 01/83725, WO 02/22776, WO 02/077218, WO 03/27147, WO 03/37932; WO 02/38162 (Scripps Research Institute); and FVIIa variants with enhanced activity as disclosed in JP 2001061479 (Chemo-Sero-Therapeutic Res Inst.).

Non-limiting examples of Factor VII variants having substantially reduced or modified biological activity relative to wild-type Factor VII include R¹⁵²E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. J. Vasc. Endovasc. Surg. 15:515-520, 1998), and Factor VIIa lacking the Gla domain, (Nicolaisen et al., FEBS Letts. 317:245-249, 1993).

Examples of Factor VII polypeptides include, without limitation, wild-type Factor VII, L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A —FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R¹⁵²E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys, and FVII having substitutions, deletions, or additions in the amino acid sequence Ile153-Arg223.

In some embodiments, the Factor VII polypeptide is human Factor VIIa (hFVIIa), preferably recombinantly made human Factor VIIa (rhVIIa).

In other embodiments, the Factor VII polypeptide is a Factor VII sequence variant.

In some embodiments, the Factor VII polypeptide has a glycosylation different from wild-type human Factor VII.

In various embodiments, e.g. those where the Factor VII polypeptide is a Factor VII-related polypeptide or a Factor VII sequence variant, the ratio between the activity of the Factor VII polypeptide and the activity of native human Factor VIIa (wild-type FVIIa) is at least about 1.25, preferably at least about 2.0, or 4.0, most preferred at least about 8.0, when tested in the “In Vitro Proteolysis Assay” (Assay 2) as described in the present specification.

In some embodiments, the Factor VII polypeptides are Factor VII-related polypeptides, in particular variants, wherein the ratio between the activity of said Factor VII polypeptide and the activity of native human Factor VIIa (wild-type FVIIa) is at least about 1.25 when tested in the “In Vitro Hydrolysis Assay” (see Assay 1 below); in other embodiments, the ratio is at least about 2.0; in further embodiments, the ratio is at least about 4.0.

In a pharmaceutical composition, it is often desirable that the concentration of the active ingredient is such that the application of a unit dose does not cause unnecessary discomfort to the patient. Thus, a unit dose of more than about 2-10 mL is often undesirable. For the purpose of the present invention, the concentration of the Factor VII polypeptide is therefore at least 0.01 mg/mL. In different embodiments, the Factor VII polypeptide is present in a concentration of 0.01-20 mg/mL; 0.1-10 mg/mL; 0.5-5.0 mg/mL; 0.6-4.0 mg/mL; 1.0-4.0 mg/mL; 0.1-5 mg/mL; 0.1-4.0 mg/mL; 0.1-2 mg/mL; or 0.1-1.5 mg/mL.

Factor VIIa concentration is conveniently expressed as mg/mL or as IU/mL, with 1 mg usually representing 43,000-56,000 IU or more.

Buffering Agent (ii)

In order to render the liquid, aqueous pharmaceutical composition useful for direct parenteral administration to a mammal such as a human, it is normally required that the pH value of the composition is held within reasonable limits, such as from about 5.0 to about 9.0. To ensure a suitable pH value under the conditions given, the pharmaceutical composition also comprises a buffering agent (ii) suitable for keeping pH in the range of from about 5.0 to about 9.0.

The term “buffering agent” encompasses those agents or combinations of agents which maintain the solution pH in an acceptable range from about 5.0 to about 9.0.

In one embodiment, the buffering agent (ii) is at least one component selected from the groups consisting of acids and salts of MES, PIPES, ACES, BES, TES, HEPES, TRIS, histidine, imidazole, glycine, glycylglycine, glycinamide, phosphoric acid, acetic acid (e.g. sodium acetate), lactic acid, glutaric acid, citric acid, tartaric acid, malic acid, maleic acid, and succinic acid. It should be understood that the buffering agent may comprise a mixture of two or more components, wherein the mixture is able to provide a pH value in the specified range. As examples can be mentioned acetic acid and sodium acetate, etc.

Due to the fact that the composition comprises very small amounts of calcium, it is possible to utilise a buffer system based on phosphoric acid, i.e. a phosphate buffer, without undesirable precipitation of calcium phosphates. Thus, in one interesting embodiment, the buffer is a phosphate buffer.

The concentration of the buffering agent is chosen so as to maintain the preferred pH of the solution. In various embodiments, the concentration of the buffering agent is 1-100 mM; 1-50 mM; 1-25 mM; or 2-20 mM.

In one embodiment, the pH of the composition is kept from about 5.0 to about 8.0; such as from about 5.0 to about 7.5; from about 5.0 and about 7.0; from about 5.0 to about 6.5, from about 5.0 to about 6.0, from about 5.5 to about 7.0; from about 5.5 to about 6.5, from about 6.0 to about 7.0, from about 6.4 to about 6.6, or from about 5.2 to about 5.7.

As used herein, pH values specified as “about” are understood to be +0.1, e.g. about pH 8.0 includes pH 8.0±0.1.

Stabilizing Agent (iii)

In a currently preferred embodiment, the composition further comprises a stabilizing agent (iii).

The stabilizing agent (iii) is, when included, typically present in a concentration of at least 5 μM, at least 25 μM, at least 50 μM, at least 100 μM, at least 200 μM, at least 400 μM, at least 500 μM, at least 800 μM, at least 900 μM, at least 1000 μM, at least 5 mM, such as 20-2000 μM, 50-5000 μM, 0.1-10 mM, 0.2-20 mM, or 0.5-50 mM.

Divalent Metal-Type Stabilizing Agent (iiia)

In one embodiment, the stabilising agent (iii) includes at least one metal-containing agent (iiia), wherein said metal is selected from the group consisting of first transition series metals of oxidation state +II.

The present inventors are under the impression that first transition series metals of oxidation state +II have not previously been utilised as stabilising agents in connection with ready-to-use pharmaceutical compositions.

When used herein, the term “first transition series metals of oxidation state +II” is intended to encompass the metals titanium, vanadium, chromium, manganese, iron, cobalt, nickel, copper, and zinc.

Although titanium and vanadium may exist in oxidation state +II in aqueous environments, it is more typical to select the metal(s) among chromium, manganese, iron, cobalt, nickel, copper, and zinc. Illustrative examples of metal-containing agents (iiia) corresponding to these metals are chromium(II) chloride, manganese(II) chloride, iron(II) chloride, cobalt(II) chloride, nickel(II) chloride, and copper(II) chloride. It should be understood that the metal-containing agent (iiia) may comprise two or more metals, e.g. two or more first transition series metals. Thus in some instances, two or more of the above-mentioned agents may be used in combination.

So far, the most promising metals are copper and manganese. Illustrative examples of corresponding metal-containing agents (iiia) are copper(II) chloride and manganese(II) chloride.

The concentration of the metal-containing agent (or agents) (iiia) is typically at least 1 μM. The desirable (or necessary) concentration typically depends on the selected metal-containing agent (or agents), more specifically on the binding affinity of the selected metal of oxidation state +II to the Factor VII polypeptide.

In different embodiments, the metal-containing agent (iiia) is present in a concentration of at least 5 μM, at least 25 μM, at least 50 μM, at least 100 μM, at least 200 μM, at least 400 μM, at least 500 μM, at least 800 μM, at least 900 μM, at least 1000 μM, at least 5 mM, at least 25 mM, at least 50 mM, at least 100 mM, at least 200 mM, at least 400 mM, at least 800 mM, at least 900 mM, or at least 1000 mM.

In one particular embodiment, the metal of the metal-containing agent (iiia) is copper and the concentration of said agent is at least 5 μM, such as at least 10 μM, or at least 15 μM.

In another particular embodiment, the metal of the metal-containing agent (iiia) is manganese and the concentration of said agent is at least 100 μM, such as at least 500 μM, or at least 1 mM.

Benzamidine/Arginine Type Stabilizing Agent (iiib)

In another embodiment, the stabilizing agent includes at least one agent (iiib) comprising a —C(═N-Z¹-R¹)—NH-Z²-R² motif, wherein
Z¹ and Z² independently are selected from the group consisting of —O—, —S—, —NRH— and a single bond, where RH is selected from the group consisting of hydrogen, C₁₋₄-alkyl, aryl and arylmethyl, and R¹ and R² independently are selected from the group consisting of hydrogen, optionally substituted C₁₋₆-alkyl, optionally substituted C₂₋₆-alkenyl, optionally substituted aryl, optionally substituted heterocyclyl, or
Z² and R² are as defined above and —C═N-Z¹-R¹ forms part of a heterocyclic ring, or Z¹ and R¹ are as defined above and —C—NH-Z²-R² forms part of a heterocyclic ring, or —C(═N-Z¹-R¹)—NH-Z²-R² forms a hetercyclic ring wherein -Z¹-R¹-R²-Z²- is a biradical. The term “C₁₋₆-alkyl” is intended to encompass acyclic and cyclic saturated hydrocarbon residues which have 1-6 carbon atoms and which can be linear or branched. Particular examples are methyl, ethyl, n-propyl, isopropyl, cyclopropyl, n-butyl, isobutyl, sec-butyl, tertbutyl, cyclopropylmethyl, n-pentyl, isopentyl, n-hexyl, etc. Similarly, the term “C₁₋₄-alkyl” encompasses acyclic and cyclic saturated hydrocarbon residues which have 1-4 carbon atoms and which can be linear or branched.

Similarly, the term “C₂₋₆-alkenyl” is intended to encompass acyclic and cyclic hydrocarbon residues which have 2-6 carbon atoms and comprise one unsaturated bond, which can be linear or branched. Examples of C₂₋₆-alkenyl groups are vinyl, allyl, but-1-en-1-yl, but-2-en-1-yl, pent-1-en-1-yl, and hex-1-en-1-yl.

The term “optionally substituted” in connection with C₁₋₆-alkyl and C₂₋₆-alkenyl groups is intended to denote that the group in question may be substituted one or several times, preferably 1-3 times, with group(s) selected from the group consisting of hydroxy, C₁₋₆-alkoxy (i.e. C₁₋₆-alkyl-oxy), C₂₋₆-alkenyloxy, oxo (forming a keto or aldehyde functionality), aryl, aryloxy, arylcarbonyl, heterocyclyl, heterocyclyloxy, heterocyclylcarbonyl, amino, mono- and di(C₁₋₆-alkyl)amino, halogen, where any aryl and heterocyclyl may be substituted as specifically described below for optionally substituted aryl and heterocyclyl.

“Halogen” includes fluoro, chloro, bromo, and iodo.

When used herein, the term “aryl” is intended to denote a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heterocyclyl” is intended to denote a saturated, partially unsaturated, partially aromatic or fully aromatic carbocyclic ring or ring system where one or more of the carbon atoms have been replaced with heteroatoms, e.g. nitrogen (═N— or —NH), sulphur (—S—), and/or oxygen (—O—) atoms. Examples of such heterocyclyl groups are oxazolyl, oxazolinyl, oxazolidinyl, isoxazolyl, isoxazolinyl, isoxazolidinyl, oxadiazolyl, oxadiazolinyl, oxadiazolidinyl, thiazolyl, isothiazolyl, pyrrolyl, pyrrolinyl, pyrrolidinyl, imidazolyl, imidazolinyl, imidazolidinyl, pyrazolyl, pyridinyl, pyrazinyl, pyridazinyl, piperidinyl, coumaryl, furyl, quinolyl, benzothiazolyl, benzotriazolyl, benzodiazolyl, benzoxozolyl, diazolyl, diazolinyl, diazolidinyl, triazolyl, triazolinyl, triazolidinyl, tetrazol, etc. Preferred heterocyclyl groups are 5-, 6- or 7-membered monocyclic groups such as isoxazolyl, isoxazolinyl, oxadiazolyl, oxadiazolinyl, pyrrolyl, pyrrolinyl, diazolyl, diazolinyl, triazolyl, triazolinyl, imidazolyl, imidazolinyl, etc.

The term “heterocyclic ring” is intended to mean a ring corresponding to those defined under “heterocyclyl”.

In connection with the terms “aryl”, “heterocyclyl” and “heterocyclic ring”, the term “optionally substituted” is intended to denote that the group in question may be substituted one or several times, preferably 1-3 times, with group(s) selected from hydroxy (which when present in an enol system may be represented in the tautomeric keto form), C₁₋₆-alkyl, C₂₋₆-alkenyl, phenyl, benzyl, C₁₋₆-alkoxy, oxo (which may be represented in the tautomeric enol form), carboxy, C₁₋₆-alkoxycarbonyl, C₁₋₆-alkylcarbonyl, amino, mono- and di(C₁₋₆-alkyl)amino, dihalogen-C₁₋₄-alkyl, trihalogen-C₁₋₄-alkyl, and halogen. The most typical examples of substituents are hydroxyl, C₁₋₄-alkyl, phenyl, benzyl, C₁₋₄-alkoxy, oxo, amino, mono- and dimethylamino and halogen.

Besides the fact that R¹ and R² independently can be selected from the group consisting of hydrogen, optionally substituted C₁₋₆-alkyl, optionally substituted C₂₋₆-alkenyl, optionally substituted aryl, optionally substituted heterocyclyl, it is also possible that a part of the —C(═N-Z¹-R¹)—NH-Z²-R² motif may be part of a hetercyclic ring, while the other part of the motif has the meaning defined for Z¹, Z², R¹ and R², respectively. In some interesting embodiments, —C═N-Z¹-R¹ may form part of a heterocyclic ring selected from the group consisting of a 1,2-diazole ring, an isoxazole ring, a 1,2,4-triazole ring, and a 1,2,4-oxadiazole ring, or —C—N H-Z²-R² may form part of a heterocyclic ring selected from the group consisting of a 1,2-diazoline ring, an isoxazoline ring, a 1,2,4-triazoline ring, and a 1,2,4-oxadiazoline ring. Such heterocyclic rings may be substituted as described above.

In some embodiments, at least one of R¹ and R² is hydrogen, e.g. both are hydrogen. Further, in some embodiment, which may be combined with the embodiments mentioned before, at least one of Z¹ and Z² is a single bond, e.g. both are a single bond. In special embodiments, R¹ and R² are both hydrogen, and Z¹ and Z² are both a single bond.

It is believed that the —C(═N-Z¹-R¹)—NH-Z²-R² motif is particularly important for the stabilising effect of the stabilising agent (iiib). In particular, it is believed that the —C(═N-Z¹-R¹)—NH-Z²-R² motif mimics an arginine moiety of a substrate for the Factor VII polypeptide.

In more specific embodiments, the stabilising agent (iiib) is at least one selected from the group consisting of amidine compounds comprising a —C—C(═N-Z¹-R¹)—NH-Z²-R² motif and guanidines compounds comprising a >N—C(═N-Z¹-R¹)—NH-Z²-R² motif. In some embodiments, the stabilising agent (iiib) is at least one amidine compound selected from the group consisting of benzamidines comprising the motif —C₆H₄—C(═N-Z¹-R¹)—NH-Z²-R², wherein C₆H₄ denotes an optionally substituted benzene ring, of which benzamidine (R¹ and R² are hydrogen and Z¹ and Z² are a single bond) constitutes a particular embodiment.

In other particular embodiments thereof, the benzamidines comprises the motif >N—C₆H₄—C(═N-Z¹-R¹)—NH-Z²-R², wherein C₆H₄ denotes an optionally substituted benzene ring, i.e. an o-amino-benzamidine, a m-amino-benzamidine or a p-amino-benzamidine, of which p-amino-benzamidines, such as p-amino-benzamidine, are the currently most promising.

Further illustrative examples of p-amino-benzamidines are those disclosed by Aventis in EP 1 162 194 A1, cf. in particular those defined in claims 1-6 and in sections [0009]-[0052], and in EP 1 270 551 A1, cf. in particular claims 1 and 2 and sections [0010]-[0032].

In another embodiment, the stabilising agent (iiib) is at least one guanidine compound selected from the group consisting of guanidines compounds comprising a —CH₂—NH—C(═N-Z¹-R¹)—NH-Z²-R² motif. Examples of guanidine compounds are those selected from the group consisting of arginine, arginine derivatives and peptides of 2-5 amino acid residues comprising at least one arginine residue. Arginine constitutes a particular embodiment.

The term “arginine derivatives” is intended to encompass arginine homologues, N-terminal functionalised arginines (e.g. N-methylated and N-acylated (e.g. acetylated) derivatives), C-terminal functionalised arginines (e.g. C-amidated, C-alkylamidated, and C-alkylated derivatives), and combinations thereof.

As mentioned above, the one crucial motif of the stabilising agents is —C(═N-Z¹-R¹)—NH-Z²-R². Other parts of the stabilising agent may also be important, in particular with respect to optimisation of the stabilising effect and the tolerance by the patient. Typically, the stabilising agent has the formula Y-C(═N-Z¹-R¹)—NH-Z²-R², wherein Y is an organic radical. The radical Y is typically selected in order to improve the efficiency of the stabilising effect. Also, the radical Y may comprise one or more further motifs of the formula —C(═N-Z¹-R¹)—NH-Z²-R².

The molecular weight of the stabilising agent is typically at the most 1000 Da, such as at the most 500 Da.

The concentration of the stabilising agent (or agents) (iiib) is typically at least 1 μM. The desirable (or necessary) concentration typically depends on the selected stabilising agent (or agents), more specifically on the binding affinity of the selected stabilising agent to the Factor VII polypeptide.

In different embodiments, the stabilising agent (iiib) is present in a concentration of at least 5 μM, at least 10 μM, at least 20 μM, at least 50 μM, at least 100 μM, at least 150 μM, at least 250 μM, at least 500 μM, at least 1 mM, at least 2 mM, at least 4 mM, at least 5 mM, at least 8 mM, at least 9 mM, at least 10 mM, at least 15 mM, at least 20 mM, such as 20-2000 μM, 50-5000 μM, 0.1-10 mM, 0.2-20 mM, or 0.5-50 mM.

In one embodiment, the stabilising agent (iiib) is benzamidine and the concentration of said agent is at least 0.5 mM, such as at least 2 mM, although it is envisaged that substituted benzamidines may be more potent for what reason they can be added in lower concentrations.

In one embodiment, the stabilising agent (iiib) is arginine and the concentration of said agent is at least 2 mM, such as at least 10 mM.

It should be understood that one or more metal-containing agents (iiia) and one or more stabilizing agents (iiib) may be used in combination.

Non-Ionic Surfactant (iv)

In another embodiment which may be combined with the above embodiments relating to the presence of a stabilizing agent (iii) (e.g. a metal-containing agent (iiia) or an agent (iiib)), the composition comprising a non-ionic surfactant (iv). Surfactants (also known as detergents) generally include those agents which protect the protein from air/solution interface induced stresses and solution/surface induced stresses (e.g. resulting in protein aggregation).

Typically, the non-ionic surfactant (iv) is at least one selected from polysorbates, poloxamers, polyoxyethylene alkyl ethers, polyethylene/polypropylene block co-polymers, polyethyleneglycol (PEG), polyoxyethylene stearates, and polyoxyethylene castor oils.

Illustrative examples of non-ionic surfactants are Tween®, polysorbate 20, polysorbate 80, Brij-35 (polyoxyethylene dodecyl ether), poloxamer 188, poloxamer 407, PEG8000, Pluronic® polyols, polyoxy-23-lauryl ether, Myrj 49, Solutol HS15, and Cremophor A.

In one embodiment, the non-ionic surfactant is present in an amount of 0.005-2.0% by weight.

Tonicity Modifying Agent—High Ionic Strength

In a further embodiment which may be combined with the above embodiments relating to the presence of a stabilizing agent (iii) (e.g. a metal-containing agent (iiia) or an agent (iiib)) and/or a non-ionic surfactant (iv), the composition may comprise a tonicity modifying agent (v).

As used herein, the term “tonicity modifying agent” includes agents which contribute to the osmolality of the solution. The tonicity modifying agent (v) includes at least one agent selected from the group consisting of neutral salts, amino acids, peptides of 2-5 amino acid residues, monosaccharides, disaccharides, polysaccharides, and sugar alcohols. In some embodiments, the composition comprises two or more of such agents in combination.

By “neutral salt” is meant a salt that is neither an acid nor a base when dissolved in an aqueous solution.

In one embodiment, at least one tonicity modifying agent (v) is a neutral salt selected from the groups consisting of sodium salts, potassium salts, and magnesium salts, such as sodium chloride, potassium chloride, magnesium chloride, magnesium acetate, magnesium gluconate, and magnesium laevulate.

In a further embodiment, the tonicity modifying agent (v) includes sodium chloride in combination with at least one selected from the groups consisting of magnesium chloride and magnesium acetate.

In a still further embodiment, the tonicity modifying agent (v) is at least one selected from the group consisting of sodium chloride, sucrose, glucose, and mannitol.

In different embodiments, the tonicity modifying agent (v) is present in a concentration of at least 1 mM, at least 5 mM, at least 10 mM, at least 20 mM, at least 50 mM, at least 100 mM, at least 200 mM, at least 400 mM, at least 800 mM, at least 1000 mM, at least 1200 mM, at least 1500 mM, at least 1800 mM, at least 2000 mM, or at least 2200 mM.

In one series of embodiments, the tonicity modifying agent (v) is present in a concentration of 5-2200 mM, such as 25-2200 mM, 50-2200 mM, 100-2200 mM, 200-2200 mM, 400-2200 mM, 600-2200 mM, 800-2200 mM, 1000-2200 mM, 1200-2200 mM, 1400-2200 mM, 1600-2200 mM, 1800-2200 mM, or 2000-2200 mM; 5-1800 mM, 25-1800 mM, 50-1800 mM, 100-1800 mM, 200-1800 mM, 400-1800 mM, 600-1800 mM, 800-1800 mM, 1000-1800 mM, 1200-1800 mM, 1400-1800 mM, 1600-1800 mM; 5-1500 mM, 25-1400 mM, 50-1500 mM, 100-1500 mM, 200-1500 mM, 400-1500 mM, 600-1500 mM, 800-1500 mM, 1000-1500 mM, 1200-1500 mM; 5-1200 mM, 25-1200 mM, 50-1200 mM, 100-1200 mM, 200-1200 mM, 400-1200 mM, 600-1200 mM, or 800-1200 mM.

In a preferred embodiment of the invention, at least one tonicity modifying agent (v) is an ionic strength modifying agent (v/a).

As used herein, the term “ionic strength modifying agent” includes agents which contribute to the ionic strength of the solution. The agents include, but are not limited to, neutral salts, amino acids, peptides of 2 to 5 amino acid residues. In some embodiments, the composition comprises two or more of such agents in combination.

Preferred examples of ionic strength modifying agents (v/a) are neutral salts such as sodium chloride, potassium chloride, and magnesium chloride. A preferred agent (v/a) is sodium chloride.

The term “ionic strength” is the ionic strength of the solution (μ) which is defined by the equation: μ=½Σ([i](Zᵢ²)), where μ is the ionic strength, [i] is the millimolar concentration of an ion, and Zᵢ is the charge (+ or −) of that ion “(see, e.g., Solomon, Journal of Chemical Education, 78(12):1691-92, 2001; James Fritz and George Schenk: Quantitative Analytical Chemistry, 1979).

In different embodiments of the invention, the ionic strength of the composition is at least 50, such as at least 75, at least 100, at least 150, at least 200, at least 250, at least 400, at least 500, at least 650, at least 800, at least 1000, at least 1200, at least 1600, at least 2000, at least 2400, at least 2800, or at least 3200.

In some specific embodiments, the total concentration of the tonicity modifying agent (v) and the ionic strength modifying agent (v/a) is in the range of 1-500 mM, such as 1-300 mM, or 10-200 mM, or 20-150 mM, depending on the effect any other ingredients may have on the tonicity and ionic strength.

In one embodiment, the composition is isotonic; in another, it is hypertonic. The term “isotonic” means “isotonic with serum”, i.e. at about 300±50 milliosmol/kg. The tonicity is meant to be a measure of osmolality of the solution prior to administration. The term “hypertonic” is meant to designate levels of osmolality above the physiological level of serum, such as levels above 300±50 milliosmol/kg.

Also, a particular embodiment of the present invention relates to the combination of the stabilising agent (iii) with a fairly high concentration of an ionic strength modifying agent (v/a) selected from the group consisting of sodium salts and magnesium salts. In this embodiment, the ionic strength modifying agent (v/a), i.e. the sodium salt and/or magnesium salt, is present in a concentration of 15-1000 mM, such as 25-1000 mM, 50-1000 mM, 100-1000 mM, 200-1000 mM, 300-1000 mM, 400-1000 mM, 500-1000 mM, 600-1000 mM, 700-1000 mM; 15-800 mM, 25-800 mM, 50-800 mM, 100-800 mM, 200-800 mM, 300-800 mM, 400-800 mM, 500-800 mM; 15-600 mM, 25-600 mM, 50-600 mM, 100-600 mM, 200-600 mM, 300-600 mM; 15-400 mM, 25-400 mM, 50-400 mM, or 100-400 mM.

Within these embodiments, sodium salt may be sodium chloride, and the magnesium salt may be selected from the group consisting of magnesium chloride, magnesium acetate, magnesium gluconate, magnesium laevulate, and magnesium salts of strong acids. In a more specific embodiment, a magnesium salt is used in combination with sodium chloride.

In one currently preferred embodiment, the composition comprises one or more ionic strength modifying agents selected from magnesium (Mg³⁺) salts, e.g. one or more salts selected from the group consisting of magnesium chloride, magnesium acetate, magnesium sulphate, magnesium gluconate, magnesium laevulate, magnesium salts of strong acids. In one embodiment hereof, the concentration of the magnesium (Mg³⁺) salt(s) is at least 2 mM, such as at least 5 mM or about 10 mM.

Other Ingredients

In addition to the above-mentioned components, the liquid, aqueous pharmaceutical composition may comprise additional components beneficial for the preparation, formulation, stability, or administration of the composition.

Thus, the composition may further comprise an antioxidant (vi). In different embodiments, the antioxidant is selected from the group consisting of L-methionine, D-methionine, methionine analogues, methionine-containing peptides, methionine-homologues, ascorbic acid, cysteine, homocysteine, gluthatione, cystine, and cysstathionine. In a preferred embodiment, the antioxidant is L-methionine.

The concentration of the antioxidant is typically 0.1-5.0 mg/mL, such as 0.1-4.0 mg/mL, 0.1-3.0 mg/mL, 0.1-2.0 mg/ml, or 0.5-2.0 mg/mL.

In particular embodiments, the composition does not include an antioxidant; instead the susceptibility of the Factor VII polypeptide to oxidation is controlled by exclusion of atmospheric air. The use of an antioxidant may of course also be combined with the exclusion of atmospheric air.

Thus, the present invention also provides an air-tight container (e.g. a vial or a cartridge (such as a cartridge for a pen applicator)) containing a liquid, aqueous pharmaceutical composition as defined herein, and optionally an inert gas. This aspect is discussed further below.

In addition to the mandatory components, the stabilizing agent (iii), the non-ionic surfactant (iv), the tonicity modifying agent (v) and the optional antioxidant (vi), the pharmaceutical composition may further comprise a preservative (vii).

A preservative may be included in the composition to retard microbial growth and thereby allow “multiple use” packaging of the Factor VII polypeptides. Examples of preservatives include phenol, benzyl alcohol, orto-cresol, meta-cresol, para-cresol, methyl paraben, propyl paraben, benzalkonium chloride, and benzethonium chloride. The preservative is normally included at a concentration of 0.1-20 mg/mL depending on the pH range and type of preservative. Still further, the composition may also include an agent capable of inhibiting deamidation and isomerization.

### PARTICULAR EMBODIMENTS

The present inventors have presently identified the following embodiments as particularly advantageous:

A liquid, aqueous pharmaceutical composition which comprises:
0.1-10 mg/mL of a Factor VII polypeptide (i);
a buffering agent (ii) suitable for keeping pH in the range of from about 5.0 to about 9.0; and
a tonicity modifying agent (v) in a concentration of at least 5 mM,
wherein the molar ratio of non-complexed calcium ions (Ca²⁺) to the Factor VII polypeptide is lower than 0.5.

A liquid, aqueous pharmaceutical composition which comprises:
0.1-10 mg/mL of a Factor VII polypeptide (i);
a buffering agent (ii) suitable for keeping pH in the range of from about 5.0 to about 9.0;
a non-ionic surfactant (iv); and a tonicity modifying agent (v) in a concentration of at least 5 mM,
wherein the molar ratio of non-complexed calcium ions (Ca²⁺) to the Factor VII polypeptide is lower than 0.5.

A liquid, aqueous pharmaceutical composition which comprises:
0.1-10 mg/mL of a Factor VII polypeptide (i);
a buffering agent (ii) suitable for keeping pH in the range of from about 5.0 to about 9.0;
a stabilizing agent (iii);
a non-ionic surfactant (iv); and
a tonicity modifying agent (v) in a concentration of at least 5 mM,
wherein the molar ratio of non-complexed calcium ions (Ca²⁺) to the Factor VII polypeptide is lower than 0.5.

A liquid, aqueous pharmaceutical composition which comprises:
0.1-10 mg/mL of a Factor VII polypeptide (i);
a buffering agent (ii) suitable for keeping pH in the range of from about 5.0 to about 9.0;
a copper-containing agent (iiia) in a concentration of at least 5 μM and/or a manganese-containing agent (iiia) in a concentration of at least 100 μM;
a non-ionic surfactant (iv); and
a tonicity modifying agent (v) in a concentration of at least 5 mM,
wherein the molar ratio of non-complexed calcium ions (Ca²⁺) to the Factor VII polypeptide is lower than 0.5.

A liquid, aqueous pharmaceutical composition which comprises:
0.1-10 mg/mL of a Factor VII polypeptide (i);
a buffering agent (ii) suitable for keeping pH in the range of from about 5.0 to about 9.0;
at least one stabilising agent (iiib) comprising the motif —C₆H₄—C(═N-Z¹-R¹)—NH-Z²-R² in a concentration of at least 5 μM and/or at least one stabilising agent (iiib) comprising the motif —CH₂—NH—C(═N-Z¹-R¹)—NH-Z²-R² in a concentration of at least 500 μM;
a non-ionic surfactant (iv); and
a tonicity modifying agent (v) in a concentration of at least 5 mM,
wherein the molar ratio of non-complexed calcium ions (Ca²⁺) to the Factor VII polypeptide is lower than 0.5.

In the above embodiments, the buffering agent preferably comprises phosphoric acid.

Properties of the Compositions of the Present Invention

The compositions according to the present invention are useful as stable and preferably ready-to-use compositions of Factor VII polypeptides. Furthermore, it is believed that the principles, guidelines and specific embodiments given herein are equally applicable for bulk storage of Factor VII polypeptides, mutatis mutandis.

The compositions are typically stable for at least six months, and preferably up to 36 months; when stored at temperatures ranging from 2° C. to 8° C. The compositions are chemically and/or physically stable, in particular chemically stable, when stored for at least 6 months at from 2° C. to 8° C.

The term “stable” is intended to denote that (i) after storage for 6 months at 2° C. to 8° C. the composition retains at least 50% of its initial biological activity as measured by a one-stage clot assay (Assay 4), or (ii) after storage for 6 months at 2° C. to 8° C., the content of heavy chain degradation products is at the most 40% (w/w) assuming that the initial sample comprises no heavy chain degradation products (i.e. only the Factor VII polypeptide is entered into the calculation of the percentage).

For the purpose of determining the biological activity as measured by a one-stage clot assay (Assay 4), the sample to be tested is diluted in 50 mM Tris (pH 7.5), 0.1% BSA and 100 μl is incubated with 100 μl of Factor VII deficient plasma and 200 μl of thromboplastin C containing 10 mM Ca²⁺. Clotting times are measured and compared to a standard curve using a reference standard or a pool of citrated normal human plasma in serial dilution.

Preferably, the stable composition retains at least 70%, such as at least 80%, or at least 85%, or at least 90%, or at least 95%, of its initial activity after storage for 6 months at 2 to 8° C.

For the purpose of determining the content of heavy chain degradation products, a reverse phase HPLC was run on a proprietary 4.5×250 mm butyl-bonded silica column with a particle size of 5 μm and pore size 300 Å. Column temperature: 70° C. A-buffer: 0.1% v/v trifluoracetic acid. B-buffer: 0.09% v/v trifluoracetic acid, 80% v/v acetonitrile. The column was eluted with a linear gradient from X to (X+13)% B in 30 minutes. X was adjusted so that FVIIa elutes with a retention time of approximately 26 minutes. Flow rate: 1.0 mL/min. Detection: 214 nm. Load: 25 μg FVIIa.

The term “physically stable” is intended to designate a composition which remains visually clear. Physical stability of the compositions is evaluated by means of visual inspection and turbidity after storage of the composition at different temperatures for various time periods. Visual inspection of the compositions is performed in a sharp focused light with a dark background. A composition is classified as physically unstable, when it shows visual turbidity.

The term “physical stability” of Factor VII polypeptides relates to the formation of insoluble and/or soluble aggregates in the form of dimeric, oligomeric and polymeric forms of Factor VII polypeptides as well as any structural deformation and denaturation of the molecule.

The term “chemically stable” is intended to designate a composition which retains at least 50% of its initial biological activity after storage for 6 months at 2 to 8° C., as measured by a one-stage clot assay (Assay 4).

The term “chemical stability” is intended to relate to the formation of any chemical change in the Factor VII polypeptides upon storage in solution at accelerated conditions. Examples are hydrolysis, deamidation, isomerisation and oxidation as well as enzymatic degradation resulting in formation of fragments of Factor VII polypeptides. In particular, the sulphur-containing amino acids are prone to oxidation with the formation of the corresponding sulphoxides.

Preparation of the Compositions of the Present Invention

In a further aspect, the invention also provides a method for preparing the liquid, aqueous pharmaceutical compositions of the invention.

Thus in one embodiment, the method for preparing a liquid, aqueous pharmaceutical composition of a Factor VII polypeptide comprises the step of providing the Factor VII polypeptide (i) in a solution comprising a buffering agent (ii) suitable for keeping pH in the range of from about 5.0 to about 9.0; while ensuring that, in the final composition, the molar ratio of non-complexed calcium ions (Ca²⁺) to the Factor VII polypeptide is lower than 0.5.

Thus in another embodiment, the method for preparing a liquid, aqueous pharmaceutical composition of a Factor VII polypeptide comprises the step of providing the Factor VII polypeptide (i) in a solution comprising a buffering agent (ii) suitable for keeping pH in the range of from about 5.0 to about 9.0; at least one metal-containing agent (iii), wherein said metal is selected from the group consisting of first transition series metals of oxidation state +II; and a non-ionic surfactant (iv); while ensuring that, in the final composition, the molar ratio of non-complexed calcium ions (Ca²⁺) to the Factor VII polypeptide is lower than 0.5.

Thus in still another embodiment, the method for preparing the liquid, aqueous pharmaceutical composition of a Factor VII polypeptide comprises the step of providing the Factor VII polypeptide at a concentration of at least 0.01 mg/mL (i) in a solution comprising a buffering agent (ii) suitable for keeping pH in the range of from about 5.0 to about 9.0; and at least one stabilising agent (iiib) comprising a —C(═N-Z¹-R¹)—NH-Z²-R² motif, wherein
Z¹ and Z² independently are selected from the group consisting of —O—, —S—, —NR^{H}— and a single bond, where R^{H} is selected from the group consisting of hydrogen, C₁₋₄-alkyl, aryl and arylmethyl, and R¹ and R² independently are selected from the group consisting of hydrogen, optionally substituted C₁₋₆-alkyl, optionally substituted C₂₋₆-alkenyl, optionally substituted aryl, optionally substituted heterocyclyl, or
Z² and R² are as defined above and —C═N-Z¹-R¹ forms part of a heterocyclic ring, or Z¹ and R¹ are as defined above and —C—NH-Z²-R² forms part of a heterocyclic ring, or —C(═N-Z¹-R¹)—NH-Z²-R² forms a hetercyclic ring wherein -Z¹-R¹-R²-Z²- is a biradical; while ensuring that, in the final composition, the molar ratio of non-complexed calcium ions (Ca²⁺) to the Factor VII polypeptide is lower than 0.5.

It should be understood that the keeping of the molar ratio of non-complexed calcium ions (Ca²⁺) to the Factor VII polypeptide lower than 0.5 can be accomplished by selecting suitable starting materials wherein the concentration of “free” (i.e. non-complexed) calcium ions is very low, or by adding a calcium chelator so as to bind calcium ions. In the latter instance, the calcium chelator is typically added in an amount approximately corresponding to the concentration of “free” calcium ions.

Methods of Use

The liquid, aqueous pharmaceutical compositions defined herein can be used in the field of medicine either as ready-to-use compositions or a bulk solutions for the preparation of ready-to-use compositions. Thus, the present invention in particular provides the liquid, aqueous pharmaceutical compositions defined herein for use as a medicament, more particular for use as a medicament for treating a Factor VII-responsive syndrome.

Consequently, the present invention also provides the use of the liquid, aqueous pharmaceutical composition as defined herein for the preparation of a medicament for treating a Factor VII-responsive syndrome, as well as a method for treating a Factor VII-responsive syndrome, the method comprising administering to a subject in need thereof an effective amount of the liquid, aqueous pharmaceutical composition as defined herein. The preparations of the present invention may be used to treat any Factor VII-responsive syndrome, such as, e.g., bleeding disorders, including those caused by clotting Factor deficiencies (e.g. haemophilia A, haemophilia B, coagulation Factor XI deficiency, coagulation Factor VII deficiency); by thrombocytopenia or von Willebrand's disease, or by clotting Factor inhibitors, and intra cerebral haemorrhage, or excessive bleeding from any cause. The preparations may also be administered to patients in association with surgery or other trauma or to patients receiving anticoagulant therapy.

The term “effective amount” is the effective dose to be determined by a qualified practitioner, who may titrate dosages to achieve the desired response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration, or other factors known to a medical practitioner.

The term “treatment” is defined as the management and care of a subject, e.g. a mammal, in particular a human, for the purpose of combating the disease, condition, or disorder and includes the administration of a Factor VII polypeptide to prevent the onset of the symptoms or complications, or alleviating the symptoms or complications, or eliminating the disease, condition, or disorder. Pharmaceutical compositions according to the present invention containing a Factor VII polypeptide may be administered parenterally to subjects in need of such a treatment. Parenteral administration may be performed by subcutaneous, intramuscular or intravenous injection by means of a syringe, optionally a pen-like syringe. Alternatively, parenteral administration can be performed by means of an infusion pump.

In important embodiments, the pharmaceutical composition is adapted to subcutaneous, intramuscular or intravenous injection according to methods known in the art. The possibly high concentration of metal ions (in particular the divalent metals ions of the metal-containing agent (iiia)) in the pharmaceutical compositions defined herein may be disadvantageous for certain groups of patients. The present invention therefore also provides a prior-to-use method for lowering the metal ion concentration in a liquid, aqueous pharmaceutical composition, wherein said method comprises the step of contacting the liquid, aqueous pharmaceutical composition defined herein with a cation-exchange material.

An example of a cation-exchange material is Chelex-100 (Fluka-Riedel/Sigma-Aldrich). The cation-exchange material, e.g. Chelex-100, is preferably contained in a sterile container, e.g. in a glass or plastic cartridge.

It is envisaged that the liquid, aqueous pharmaceutical composition is contacted with the cation-exchange material, e.g. by passage through a cartridge containing the cation-exchange material, immediately prior to use. In a particular embodiment, it is envisaged that the cartridge is an integral part of a syringe assembly.

Suitable Container for the Pharmaceutical Composition

As mentioned above, the present invention also provides an air-tight container (e.g. a vial or a cartridge (such as a cartridge for a pen applicator)) containing a liquid, aqueous pharmaceutical composition as defined herein, and optionally an inert gas. The inert gas may be selected from the group consisting of nitrogen, argon, etc. The container (e.g. vial or cartridge) is typically made of glass or plastic, in particular glass, optionally closed by a rubber septum or other closure means allowing for penetration with preservation of the integrity of the pharmaceutical composition. In a particular embodiment hereof, the composition does not comprise a preservative (vii). In a further embodiment, the container is a vial or cartridge enclosed in a sealed bag, e.g. a sealed plastic bag, such as a laminated (e.g. metal (such as aluminium) laminated plastic bag). More particularly, the air-tight, at least partially filled container contains a liquid, aqueous pharmaceutical composition as defined herein, and optionally an inert gas, said container comprising (i) a wall portion and (ii) one or more closure means not constituting part of said wall portion. Preferably, the pharmaceutical composition does not comprise a preservative (vii).

In particular, the container inner wall material is a material selected from the group consisting of silica-coated glass, silicone-coated glass, polymers of non-cyclic olefins, cycloolefin polymers, and cycloolefin/linear olefin copolymers.

In one variant, the inner wall of a container includes various grades/types of glass to which a coating of silica (silicon dioxide, SiO₂) has been applied; one such material which is very well suited is so-called “Type I” glass (as defined in the European Pharmacopeia, Ph. Eur.) coated with silica. For the definition of, and characterizing tests for, Type I glass and other types of pharmaceutically applicable glass (Types II, III and IV), see, e.g., section 3.2.1 of Ph. Eur. at the following WorldWideWeb address: http://online.pheur.org/404TER/ep404.dll?f=templates&fn=main-h.htm&2.0.

Type I glass containers are described in section 3.2.1 of Ph. Eur. (4^{th} Edition, online) as follows: “They are of neutral glass and have a high hydrolytic resistance due to the chemical composition of the glass itself.”, neutral glass being defined therein as follows: “Neutral glass is a borosilicate glass containing significant amounts of boric oxide, aluminium or alkaline earth oxides. Due to its composition neutral glass has a high thermal shock resistance and a very high hydrolytic resistance.”

The silica coating on the inner wall of a container of this type will preferably have a substantially uniform thickness of at least about 0.05 μm, although a substantially uniform thickness in the range of from about 0.1 μm to about 0.2 μm is believed to be generally more desirable. Chemical Vapour Deposition (CVD) appears to be a technique which is very well suited for applying such a substantially uniformly thick coating of silica to glass surfaces, and Type I glass containers (e.g. vials) in which a silica coating which has been deposited by a CVD technique on the inner surface of the container, and which are very suitable for use in the context of the invention, are available commercially, e.g. Schott Type I plus™ containers from Schott Glaskontor, Mullheim/Baden, Germany. Reference may be made, for example, to the following article on the WorldWideWeb for a description of CVD techniques: http://www.azom.com/details.asp?ArticleID=1552.

As also indicated above, further preferred materials for the inner wall of a container include various grades/types of glass which—normally after initial washing or steeping in water or another aqueous medium to remove water-leachable substances or species—have been coated with a silicone. As before, a preferred type of glass in this connection is a Type I glass (Ph. Eur.).

The term “silicone” is used broadly herein to denote not only silicones per se, which typically are polymeric dialkylated, diarylated or monoalkylated+monoarylated siloxanes, but also copolymers, typically block and graft copolymers comprising silicone segments and segments of other polymeric materials such as polystyrene, polyolefins, polyamides or polyurethane.

The coating material may suitably be a poly(dialkyl-siloxane) oil or copolymer, and suitable types of poly(dialkyl-siloxane) which in this connection include poly(dimethylsiloxane) (PDMS), poly(dipropyl-siloxane) and poly(dihexyl-siloxane).

The viscosity of the oil when applied to the component may be of importance, especially for the elimination of the slip-stick phenomenon which may arise, for example, when the container in question is a cartridge or the like comprising a displaceable plunger used to expel liquid (protein formulation) from the container. The more viscous, the lesser the risk of a slip-stick phenomenon whereby smooth movement of the plunger is impeded. In one embodiment, coating comprises a linear or branched hydrophilized poly(dialkyl-siloxane) oil. The viscosity of the oil is preferably above 200,000 centistokes, such as above 500,000 centistokes when applied to the component.

The silicone coating may also comprise a cross-linked or gelled silicone oil, such as a hydrophilized poly(dialkyl-siloxane) oil, or a mixture of a cross-linked and a non-cross-linked oil. By using a cross-linked or gelled oil, the migration ability of the oil is significantly reduced, and the coating may be regarded as a solid material.

A cross-linked, or cured, silicone oil is typically obtained by applying a linear, or branched, silicone oil with reactive functionalities which are used to cross-link the coating in a subsequent step. There are a number of different available cross-linking methods, e.g. curing by irradiation with UV light, curing by heating at elevated temperature, and curing in the presence of water. A cross-linked silicone oil may also be obtained by first applying a linear or branched-chain silicone oil, and then irradiating the oil with a high-energy radiation source, e.g. an electron source or X-ray source. The cross-linkable silicone oil may suitably be one of medical grade, e.g. MDX™ supplied by Dow Corning (MDX4-4159 Fluid); other suitable types include Wacker E2 silicone oil, supplied as an approx. 35% aqueous emulsion.

In another embodiment, the silicone coating comprises a hydrophilized poly(dialkyl-siloxane) block and graft copolymer. The copolymer may be any block and graft copolymer which comprises polymeric segments of poly(dialkyl-siloxane), such as PDMS. The polymeric segments may, for example, be combined with polymeric segments of polystyrene, polyolefins, polyamides or polyurethane to form the desired copolymer. The copolymer may be prepared by any suitable known method, for example by sequential anionic polymerization, or various grafting procedures.

Hydrophilicity of a silicone coating may be achieved by any appropriate method, e.g. by subjecting the coating to an oxidative treatment, such as plasma treatment or corona treatment, after having been applied to the glass surface. Hydrophilicity may also be achieved by end-capping a copolymer with hydrophilic group or chain segments. The hydrophilic group may, for example, be a negatively charged chemical group or phosphorylcholine (PC) groups, and the chain segment may, for example, be poly(ethylene oxide) (PEO) or poly(2-hydroxyethyl methacrylate) (pHEMA).

Plasma-treated surfaces may be modified in order to decrease protein adsorption by coupling of hydrophilic polymer segments or functional groups. These polymer segments or functional groups may be of the same kind as those described above, and may further be coupled to the functional groups generated during the plasma treatment.

The thickness of the silicone coating depends on the specific coating, and is preferably from 0.005 to 10 μm, more preferably from 0.01 to 1 μm. The optimal thickness depends on the dimensions, shape and type of the container, and can easily be determined by one skilled in the art. In the case, for example, of a cartridge with a displaceable plunger or piston part, if the coating is too thin it may be torn in use, thereby increasing the friction between the plunger and the wall part. When the thickness of the coating has reached a certain plateau value the friction forces are approximately constant, even when the thickness is further increased. For any coating composition the coating should preferably be as thin as possible to reduce costs. Such a thin coating may suitably have a thickness from 0.005 to 0.4 μm, such as from 0.015 to 0.25 μm, more preferably about 0.2 μm.

Depending on the migration ability of the silicone coating the hydrophilic groups at the coating will tend to seek into the coating leaving the surface hydrophobic due to the hydrophobicity of the surrounding air. In the case of a container which is to be filled with the liquid, aqueous pharmaceutical composition of a Factor VII polypeptide, it is therefore desirable—in order to minimize any tendency of the protein in a aqueous liquid formulation thereof to adsorb to the inner container surface—that the coating remains hydrophilic during storage until the liquid protein formulation has been introduced into the container. This is most simply achieved by filling the container with the protein formulation shortly after the coating process has taken place.

As indicated above, further preferred materials for the inner wall of a container in the context of the present invention include polymers of non-cyclic (i.e. straight- or branched-chain) olefins, i.e. polyalkenes. Among such materials, useful polymers derived from a single monomer include polyethylenes and polypropylenes, numerous grades of which are partially crystalline in structure. Copolymers of non-cyclic olefins [e.g. copolymers of ethylene (ethene) and propylene (propene)] are likewise of interest as inner-wall materials in the context of the invention.

As also indicated above, further preferred materials for the inner wall of a container include cycloolefin polymers, and suitable types thereof include those consisting of substantially 100% of 5-7 membered aliphatic cyclic hydrocarbon rings. Suitable commercially available containers made of cycloolefin polymer material include containers manufactured from CZ™ resin, available from Dalkyo Seiko Ltd., Tokyo, Japan. Other relevant polymer materials of this type include Zeonor™ and Zeonex™, both from Nippon Zeon Co. Ltd. Tokyo, Japan.

Suitable types of cycloolefin/linear olefin copolymers include materials with an amorphous structure, such as the highly transparent copolymers of the Topas™ type (obtainable from Ticona GmbH, Frankfurt am Main, Germany), which are available in a variety of grades (e.g. Topas™ 8007, Topas™ 5013, Topas™ 6013, Topas™ 6015 and Topas™ 6017).

In one variant, the container having as a container inner wall material a solid-phase material which, when incubated for at least 24 months at a temperature not exceeding 40° C. in contact with water or an aqueous solution having a pH of from about 3 to about 8 releases at most about 3 μM of a trivalent metal ion into solution; the container comprising (i) a wall portion and (ii) one or more closure means not constituting part of the wall portion.

Although it is believed (as indicated above) that an acceptable upper limit for the released level/concentration of trivalent metal ions is about 3 μM (i.e. released level <about 3 μM), a released level of at most about 2.5 μM (i.e. <about 2.5 μM), more desirably at most about 1 μM (i.e. <about 1 μM), such as at most about 0.5 μM (i.e. <about 0.5 μM), appears to be advantageous.

With regard to trivalent metal ions in the context of the latter two aspects of the present invention, release of Al³⁺ appears to be particularly undesirable; Fe³⁺ constitutes a further example of a trivalent metal ion whose release into solution is to be avoided. In addition to avoidance of release of trivalent metal ions into solution, it is further believed to be desirable to avoid release into solution of certain divalent metal ions, particularly Zn²⁺. In this connection, released levels should probably not exceed about 3 μM (i.e. released level ≦ about 3 μM), more preferably about 1 μM (i.e. released level ≦ about 1 μM), such as at most about 0.5 μM (i.e. ≦ about 0.5 μM).

It may be mentioned at this point that although coated glass materials, notably silica-coated glass (notably silica-coated Type I glass) and silicone-coated glass (notably silicone-coated Type I glass), are among preferred inner-wall materials in the context of various aspects of the invention, it may—in order to comply with the criteria set forth above with regard to release of trivalent or divalent ions into solution—in some embodiments be sufficient to employ a glass, particularly a Type I (Ph. Eur.) glass, which has been subjected to a washing or extraction treatment which reduces the level of extractable trivalent and divalent metal ions present in/on the surface of the glass. Such treatments include steeping in (extraction with) hot (preferably at least 90° C.) water or another aqueous medium, e.g. ammonium sulfate solution.

In a further embodiment, said container is a vial or cartridge comprising a closure means which comprises a needle-penetrable, self-sealing elastomeric septum. In particular, the container is a cartridge further comprising a displaceable piston means whereby liquid present in said container may be expelled from said container.

### EXPERIMENTALS

General Methods

Percentages are (weight/weight) both when referring to solids dissolved in solution and liquids mixed into solutions. For example, for Tween, it is the weight of 100% stock/weight of solution.

Assays Suitable for Determining Biological Activity of Factor VII Polypeptides

Factor VII polypeptides useful in accordance with the present invention may be selected by suitable assays that can be performed as simple preliminary in vitro tests. Thus, the present specification discloses a simple test (entitled “In Vitro Hydrolysis Assay”) for the activity of Factor VII polypeptides.

In Vitro Hydrolysis Assay (Assay 1)

Native (wild-type) Factor VIIa and Factor VII polypeptide (both hereinafter referred to as “Factor VIIA”) may be assayed for specific activities. They may also be assayed in parallel to directly compare their specific activities. The assay is carried out in a microtiter plate (MaxiSorp, Nunc, Denmark). The chromogenic substrate D-Ile-Pro-Arg-p-nitroanilide (S-2288, Chromogenix, Sweden), final concentration 1 mM, is added to Factor VIIa (final concentration 100 nM) in 50 mM HEPES, pH 7.4, containing 0.1 M NaCl, 5 mM CaCl₂ and 1 mg/mL bovine serum albumin. The absorbance at 405 nm is measured continuously in a SpectraMax™ 340 plate reader (Molecular Devices, USA). The absorbance developed during a 20-minute incubation, after subtraction of the absorbance in a blank well containing no enzyme, is used for calculating the ratio between the activities of Factor VII polypeptide and wild-type Factor VIIa:
Ratio=(A405 nm Factor VII polypeptide)/(A405 nm Factor VIIa wild-type).

Based thereon, Factor VII polypeptides with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

The activity of the Factor VII polypeptides may also be measured using a physiological substrate such as Factor X (“In Vitro Proteolysis Assay”), suitably at a concentration of 100-1000 nM, where the Factor Xa generated is measured after the addition of a suitable chromogenic substrate (eg. S-2765). In addition, the activity assay may be run at physiological temperature.

In Vitro Proteolysis Assay (Assay 2)

Native (wild-type) Factor VIIa and Factor VII polypeptide (both hereinafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 μL 50 mM HEPES, pH 7.4, containing 0.1 M NaCl, 5 mM CaCl₂ and 1 mg/mL bovine serum albumin, are incubated for 15 min. Factor X cleavage is then stopped by the addition of 50 μL 50 mM HEPES, pH 7.4, containing 0.1 M NaCl, 20 mM EDTA and 1 mg/mL bovine serum albumin. The amount of Factor Xa generated is measured by the addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax™ 340 plate reader (Molecular Devices, USA). The absorbance developed during 10 minutes, after subtraction of the absorbance in a blank well containing no FVIIa, is used for calculating the ratio between the proteolytic activities of Factor VII polypeptide and wild-type Factor VIIa:
Ratio=(A405 nm Factor VII polypeptide)/(A405 nm Factor VIIa wild-type).

Based thereon, Factor VII polypeptide with an activity lower than, comparable to, or higher than native Factor VIIa may be identified, such as, for example, Factor VII polypeptides where the ratio between the activity of the Factor VII polypeptide and the activity of native Factor VII (wild-type FVII) is about 1.0 versus above 1.0.

Thrombin Generation Assay (Assay 3)

The ability of Factor VIIa or Factor VII polypeptides to generate thrombin can also be measured in an assay (Assay 3) comprising all relevant coagulation Factors and inhibitors at physiological concentrations (minus Factor VIII when mimicking hemophilia A conditions) and activated platelets (as described on p. 543 in Monroe et al. (1997) Brit. J. Haematol. 99, 542-547, which is hereby incorporated herein as reference).

One-Stage Coagulation Assay (Assay 4)

The biological activity of the Factor VII polypeptides may also be measured using a one-stage coagulation assay (Assay 4). For this purpose, the sample to be tested is diluted in 50 mM PIPES-buffer (pH 7.5), 0.1% BSA and 40 μl is incubated with 40 μl of Factor VII deficient plasma and 80 μl of human recombinant tissue factor containing 10 mM Ca2+ and synthetic phospholipids. Coagulation times are measured and compared to a standard curve using a reference standard in a parallel line assay.

Preparation and Purification of Factor VII Polypeptides

Human purified Factor VIIa suitable for use in the present invention is preferably made by DNA recombinant technology, e.g. as described by Hagen et al., Proc. Natl. Acad. Sci. USA 83: 2412-2416, 1986, or as described in European Patent No. 0 200 421 (ZymoGenetics, Inc.).

Factor VII may also be produced by the methods described by Broze and Majerus, J. Biol. Chem. 255 (4): 1242-1247, 1980 and Hedner and Kisiel, J. Clin. Invest. 71: 1836-1841, 1983. These methods yield Factor VII without detectable amounts of other blood coagulation Factors. An even further purified Factor VII preparation may be obtained by including an additional gel filtration as the final purification step. Factor VII is then converted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be activated by passing it through an ion-exchange chromatography column, such as Mono Q® (Pharmacia fine Chemicals) or the like, or by autoactivation in solution.

Factor VII-related polypeptides may be produced by modification of wild-type Factor VII or by recombinant technology. Factor VII-related polypeptides with altered amino acid sequence when compared to wild-type Factor VII may be produced by modifying the nucleic acid sequence encoding wild-type Factor VII either by altering the amino acid codons or by removal of some of the amino acid codons in the nucleic acid encoding the natural Factor VII by known means, e.g. by site-specific mutagenesis.

It will be apparent to those skilled in the art that substitutions can be made outside the regions critical to the function of the Factor VIIa molecule and still result in an active polypeptide. Amino acid residues essential to the activity of the Factor VII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al.,-1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a supercoiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than 10% by weight, more preferably less than 5% and most preferably less than 1%, of non-Factor VII polypeptides derived from the host cell.

Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII polypeptides may be activated by passing it through an ion-exchange chromatography column, such as Mono Q® (Pharmacia) or the like, or by autoactivation in solution. The resulting activated Factor VII polypeptide may then be formulated and administered as described in the present application.

The following examples illustrate practice of the invention. These examples are included for illustrative purposes only and are not intended in any way to limit the scope of the invention claimed.

### WORKING EXAMPLES

### Example 1

### Effect of Content of Calcium in Aqueous rFVIIa Solutions on Heavy Chain Degradation (Autocatalytic Cleavage)

In order to investigate the effect of calcium ions on rFVIIa, the following procedure was followed:

rFVIIa (M_{w} approx. 50,000) was transferred to the following solutions by desalting on a PD-10 column (Amersham Biosciences):

| Formulation 1-1: | | |
|---|---|---|
| rFVIIa | 1.0 | mg/mL |
| PIPES-di-Na | 17.32 | mg/mL (50 mM) |
| 1 M NaOH or 1 M HCl | added to pH 6.5 | |
| Ca²⁺/FVII ratio | 0 | |
| Formulation 1-2: | | |
| rFVIIa | 1.0 | mg/mL |
| Calcium chloride 2 H₂O | 1.47 | mg/mL (10 mM) |
| Sodium chloride | 2.92 | mg/mL (50 mM) |
| Glycylglycine | 1.32 | mg/mL (10 mM) |
| Sodium acetate | 0.82 | mg/mL (10 mM) |
| Histidine | 1.55 | mg/mL (10 mM) |
| 1 M NaOH or 1 M HCl | added to pH 6.5 | |
| Ca²⁺/FVII ratio | 500 | |

The formulations were stored at a temperature of 5° C. or 25° C., respectively, and analyses were performed at the times indicated in Table 1.

TABLE 1
Content of Heavy chain degradation
products (%) in rFVIIa formulations

| | T = 0 | T = 1 month | T = 2 months | T = 3 months |
|---|---|---|---|---|
| Formulation | 9.6 | 9.8 | 9.8 | 10.6 |
| 1-1, 5° C. | | | | |
| Formulation | 10.2 | 17.0 | 23.8 | 30.4 |
| 1-2, 5° C. | | | | |
| Formulation | 9.6 | 9.7 | 9.7 | 10.3 |
| 1-1, 25° C. | | | | |
| Formulation | 10.2 | 18.5 | 23.5 | n.d. |
| 1-2, 30° C. | | | | |

As it can be seen from Table 1, the increase in the content of Heavy chain degradation products in Formulation 1-2 was much higher than the increase for Formulation 1-1.

The content of heavy chain degradation products was determined by RP-HPLC as described in the following:

Reverse phase HPLC was run on a proprietary 4.5×250 mm butyl-bonded silica column with a particle size of 5 μm and pore size 300 Å. Column temperature: 70° C. A-buffer: 0.1% v/v trifluoracetic acid. B-buffer: 0.09% v/v trifluoracetic acid, 80% v/v acetonitrile. The column was eluted with a linear gradient from X to (X+13)% B in 30 minutes. X was adjusted so that FVIIa elutes with a retention time of approximately 26 minutes. Flow rate: 1.0 mL/min. Detection: 214 nm. Load: 25 μg FVIIa.

### Example 2

### Effect of Content of Calcium and Divalent Metal Ions in Aqueous rFVIIa Solutions on Heavy Chain Degradation (Autocatalytic Cleavage)

In order to investigate the effect of calcium ions and divalent metal ions on rFVIIa, the following procedure was followed:

rFVIIa was transferred to the following solutions by desalting on a PD-10 column (Amersham Biosciences):

All formulations (2-1 to 2-8) included

| | |
|---|---|
| rFVIIa | 1.0 mg/mL |
| Calcium chloride 2 H₂O | 1.47 mg/mL (10 mM) |
| Sodium chloride | 2.92 mg/mL (50 mM) |
| Glycylglycine | 1.32 mg/mL (10 mM) |
| Histidine | 1.55 mg/mL (10 mM) |
| 1 M NaOH or 1 M HCl | added to pH 6.5 |

and further included benzamidine and EDTA as shown in Table 2

TABLE 2

| | | | Non-complexed | |
|---|---|---|---|---|
| Formulation | Benzamidine | EDTA | calcium ions | Ca²⁺/rFVIIa |
| No. | (mM) | (mM) | (mM) | ratio |
| 2-1 | 10 | 0 | about 10 | 500 |
| 2-2 | 10 | 9.9 | about 0.1 | 5 |
| 2-3 | 10 | 15 | about 0.0 | 0.0 |
| 2-4 | 1 | 0 | about 10 | 500 |
| 2-5 | 1 | 9.9 | about 0.1 | 5 |
| 2.6 | 1 | 15 | about 0.0 | 0.0 |
| 2.7 | 0 | 0 | about 10 | 500 |

The formulations were stored at a temperature of 5° C. and analyses were performed at the times indicated in Table 3.

TABLE 3
Content of Heavy chain degradation
products (%) in rFVIIa formulations

| | | T = 2 | T = 3 | T = 4 | T = 8 |
|---|---|---|---|---|---|
| | T = 0 | weeks | weeks | weeks | weeks |
| Formulation 2-1 | 7.2 | 8.0 | 8.9 | — | 9.9 |
| Formulation 2-2 | 7.3 | 7.5 | 7.8 | — | 8.2 |
| Formulation 2-3 | 7.3 | 7.3 | 7.6 | — | 7.9 |
| Formulation 2-4 | 7.5 | 10.7 | 12.8 | — | 19.3 |
| Formulation 2-5 | 7.4 | 8.2 | 8.7 | — | 10.3 |
| Formulation 2-6 | 7.2 | 7.7 | 8.1 | — | 8.7 |
| Formulation 2-7 | 8.1 | — | — | 16.3 | — |

As it can be seen from Table 2, the content of non-complexed calcium ions had a significant influence on the heavy chain degradation of rFVII. The results also show that the stabilizing agent (benzamidine) was a more efficient stabilizer when the concentration of non-complexed calcium ions was lowered. Thus, it was estimated that about the same stability could be obtained upon concurrent reduction of the concentration of benzamidine and the concentration of non-complexed calcium ions (compare Formulations 2-3 and 2-6).

The content of heavy chain degradation products was determined by RP-HPLC as described in Example 1.

1. A liquid, aqueous pharmaceutical composition comprising
a Factor VII polypeptide (i) and
a buffering agent (ii) suitable for keeping pH in the range of from about 5.0 to about 9.0;
wherein the molar ratio of non-complexed calcium ions (Ca²⁺) to the Factor VII polypeptide is lower than 0.5.

2. The composition according to claim 1, wherein the molar ratio of non-complexed calcium ions (Ca²⁺) to the Factor VII polypeptide is in the range of 0.001-0.499.

3. The composition of claim 1, further comprising a stabilizing agent (iii).

4. The composition according to claim 3, wherein the stabilising agent (iii) includes at least one metal-containing agent (iiia), wherein said metal is selected from the group consisting of first transition series metals of oxidation state +II.

5. The composition according to claim 4, wherein the metal of the metal-containing agent is selected from the group consisting of chromium, manganese, iron, cobalt, nickel, copper, and zinc.

6. The composition according to claim 4, wherein the metal-containing agent (iiia) is at least one selected from the group consisting of chromium(II) chloride, manganese(II) chloride, iron(II) chloride, cobalt(II) chloride, nickel(II) chloride, and copper(II) chloride.

7. The composition according to claim 4, wherein the metal of the metal-containing agent (iiia) is selected from the group consisting of copper and manganese.

8. The composition according to claim 7, wherein the metal-containing agent (iiia) is selected from the group consisting of copper(II) chloride and manganese(II) chloride.

9. The composition according to claim 4, wherein the concentration of the metal-containing agent (iiia) is at least 1 μM.

10. The composition according to claim 4, wherein the metal of the metal-containing agent (iiia) is copper and the concentration of said agent is at least 5 μM.

11. The composition according to claim 4, wherein the metal of the metal-containing agent (iiia) is manganese and the concentration of said agent is at least 100 μM.

12. The composition according to claim 3, wherein the stabilizing agent includes at least one agent (iiib) comprising a —C(═N-Z¹-R¹)—NH-Z²-R² motif, wherein
Z¹ and Z² independently are selected from the group consisting of —O—, —S—, —NRH— and a single bond, where RH is selected from the group consisting of hydrogen, C₁₋₄-alkyl, aryl and arylmethyl, and R¹ and R² independently are selected from the group consisting of hydrogen, optionally substituted C₁₋₆-alkyl, optionally substituted C₂₋₆-alkenyl, optionally substituted aryl, optionally substituted heterocyclyl, or
Z² and R² are as defined above and —C═N-Z¹-R¹ forms part of a heterocyclic ring, or Z¹ and R¹ are as defined above and —C—NH-Z²-R² forms part of a heterocyclic ring, or —C(═N-Z¹-R¹)—NH-Z²-R² forms a heterocyclic ring wherein -Z¹-R¹-R²-Z²- is a biradical.

13. The composition according to claim 12, wherein at least one of R¹ and R² is hydrogen.

14. The composition according to claim 12, wherein at least one of Z¹ and Z² is a single bond.

15. The composition according to claim 12, wherein R¹ and R² are both hydrogen and Z¹ and Z² are both a single bond.

16. The composition according to claim 12, wherein the stabilising agent (iiib) is at least one selected from the group consisting of amidine compounds comprising a —C—C(═N-Z¹-R¹)—NH-Z²-R² motif and guanidines compounds comprising a >N—C(═N-Z¹-R¹)—NH-Z²-R² motif.

17. The composition according to claim 16, wherein the stabilising agent (iiib) is at least one amidine compound selected from the group consisting of benzamidines comprising the motif —C₆H₄—C(═N-Z¹-R¹)—NH-Z²-R², wherein C₆H₄ denotes an optionally substituted benzene ring.

18. The composition according to claim 17, wherein the benzamidines comprises the motif >N—C₆H₄—C(═N-Z¹-R¹)—NH-Z²-R², wherein C₆H₄ denotes an optionally substituted benzene ring.

19. The composition according to claim 16, wherein the stabilising agent (iiib) is at least one guanidine compound selected from the group consisting of guanidines compounds comprising a —CH₂—NH—C(═N-Z¹-R¹)—NH-Z²-R² motif.

20. The composition according to claim 19, wherein the guanidine compounds are selected from the group consisting of arginine, arginine derivatives, and peptides of 2-5 amino acid residues comprising at least one arginine residue.

21. The composition according to claim 12, wherein the stabilising agent has the formula Y-C(═N-Z¹-R¹)—NH-Z²-R², wherein Y is an organic radical.

22. The composition according to claim 12, wherein the molecular weight of the stabilising agent is at the most 1000 Da.

23. The composition according to claim 12, wherein the concentration of the stabilising agent (iiib) is at least 1 μM.

24. The composition according to claim 23, wherein the stabilising agent (iiib) is benzamidine and the concentration of said agent is at least 0.5 mM.

25. The composition according to claim 23, wherein the stabilising agent (iiib) is arginine and the concentration of said agent is at least 2 mM.

26. The composition of claim 1, wherein the composition further comprises a non-ionic surfactant (iv).

27. The composition according to claim 26, wherein the non-ionic surfactant (iv) is at least one selected from the group consisting of polysorbates, poloxamers, polyoxyethylene alkyl ethers, polyethylene/polypropylene block co-polymers, polyethyleneglycol (PEG), polyoxyethylene stearates, and polyoxyethylene castor oils.

28. The composition according to claim 27, wherein the non-ionic surfactant is present in an amount of 0.005-2.0% by weight.

29. The composition according to claim 1, wherein the composition further comprises a tonicity modifying agent (v).

30. The composition according to claim 29, wherein the tonicity modifying agent (v) is at least one selected from the group consisting of neutral salts, amino acids, peptides of 2-5 amino acid residues, monosaccharides, disaccharides, polysaccharides, and sugar alcohols.

31. The composition according to claim 30, wherein at least one tonicity modifying agent (v) is a neutral salt selected from the group consisting of sodium salts, potassium salts, and magnesium salts.

32. The composition according to claim 30, wherein the tonicity modifying agent (v) is sodium chloride in combination with at least one selected from the group consisting of magnesium chloride and magnesium acetate.

33. The composition according to claim 30, wherein the tonicity modifying agent (v) is present in a concentration of at least 1 mM.

34. The composition according to claim 29, wherein at least one tonicity modifying agent (v) is an ionic strength modifying agent (v/a).

35. The composition according to claim 1, wherein the composition has an ionic strength of at least 50.

36. The composition according to claim 35, wherein the composition has an ionic strength of at least 200.

37. The composition according to claim 36, wherein the composition has an ionic strength of at least 400.

38. The composition according to claim 1, wherein the composition has an osmolality of 300±50 milliosmol/kg.

39. The composition according to claim 1, wherein the buffering agent (ii) comprises at least one component selected from the group consisting of acids and salts of MES, PIPES, ACES, BES, TES, HEPES, TRIS, histidine, imidazole, glycine, glycylglycine, glycinamide, phosphoric acid, acetic acid, lactic acid, glutaric acid, citric acid, tartaric acid, malic acid, maleic acid, and succinic acid.

40. The composition according to claim 39, wherein the concentration of the buffering agent (ii) is 1-100 mM.

41. The composition according to claim 1, wherein the composition has a pH in the range of from about 5.0 to about 8.0.

42. The composition of claim 1, wherein the composition further comprises an antioxidant (vi).

43. The composition according to claim 42, wherein the antioxidant (vi) is selected from L-methionine, D-methionine, methionine analogues, methionine-containing peptides, methionine-homologues, ascorbic acid, cysteine, homocysteine, gluthatione, cystine, and cysstathionine.

44. The composition according to claim 43, wherein the antioxidant (vi) is present in a concentration of 0.1-5.0 mg/mL.

45. The composition according to claim 1, wherein the composition further comprises a preservative (vii).

46. The composition according to claim 45, wherein the preservative (vii) is selected from the group consisting of phenol, benzyl alcohol, orto-cresol, meta-cresol, para-cresol, methyl paraben, propyl paraben, benzalkonium chloride, and benzaethonium chloride.

47. The composition according to claim 1, wherein the Factor VII polypeptide is human Factor VIIa.

48. The composition according to claim 1, wherein the Factor VII polypeptide is a Factor VII sequence variant.

49. The composition according to claim 48, wherein the ratio between the activity of the Factor VII polypeptide and the activity of native human Factor VIIa (wild-type FVIIa) is at least 1.25 when tested in the “In Vitro Proteolysis Assay.”

50. The composition according to claim 1, wherein the Factor VII polypeptide is present in a concentration of 0.1-10 mg/mL.

51. The composition according to claim 1, wherein the composition comprises:
0.1-10 mg/mL of a Factor VII polypeptide (i);
a buffering agent (ii) suitable for keeping pH in the range of from about 5.0 to about 9.0; and
a tonicity modifying agent (v) in a concentration of at least 5 mM,
wherein the molar ratio of non-complexed calcium ions (Ca²⁺) to the Factor VII polypeptide is lower than 0.5.

52. The composition of claim 1, wherein the composition comprises:
0.1-10 mg/mL of a Factor VII polypeptide (i);
a buffering agent (ii) suitable for keeping pH in the range of from about 5.0 to about 9.0;
a non-ionic surfactant (iv); and
a tonicity modifying agent (v) in a concentration of at least 5 mM,
wherein the molar ratio of non-complexed calcium ions (Ca²⁺) to the Factor VII polypeptide is lower than 0.5.

53. The composition according to claim 1, wherein the composition comprises: 0.1-10 mg/mL of a Factor VII polypeptide (i);
a buffering agent (ii) suitable for keeping pH in the range of from about 5.0 to about 9.0;
a copper-containing agent (iiia) in a concentration of at least 5 μM and/or a manganese-containing agent (iiia) in a concentration of at least 100 μM;
a non-ionic surfactant (iv); and
a tonicity modifying agent (v) in a concentration of at least 5 mM,
wherein the molar ratio of non-complexed calcium ions (Ca²⁺) to the Factor VII polypeptide is lower than 0.5.

54. The composition according to claim 1, wherein the composition comprises: 0.1-10 mg/mL of a Factor VII polypeptide (i);
a buffering agent (ii) suitable for keeping pH in the range of from about 5.0 to about 9.0;
at least one stabilising agent (iiib) comprising the motif —C₆H₄—C(═N-Z¹-R¹)—NH-Z²-R² in a concentration of at least 5 μM and/or at least one stabilising agent (iiib) comprising the motif —CH₂—NH—C(═N-Z¹-R¹)—NH-Z²-R² in a concentration of at least 500 μM;
a non-ionic surfactant (iv); and
a tonicity modifying agent (v) in a concentration of at least 5 mM,
wherein the molar ratio of non-complexed calcium ions (Ca²⁺) to the Factor VII polypeptide is lower than 0.5.

55. The composition according to claim 1, wherein the composition is adapted for parenteral administration.

56. The composition according to claim 55, wherein the composition is adapted for subcutaneous, intramuscular or intravenous injection.

57. A method for preparing a liquid, aqueous pharmaceutical composition of a Factor VII polypeptide comprising the step of providing the Factor VII polypeptide (i) in a solution comprising a buffering agent (ii) suitable for keeping pH in the range of from about 5.0 to about 9.0; while ensuring that, in the final composition, the molar ratio of non-complexed calcium ions (Ca²⁺) to the Factor VII polypeptide is lower than 0.5.

58. The method according to claim 57, wherein the method comprises the step of providing the Factor VII polypeptide (i) in a solution comprising a buffering agent (ii) suitable for keeping pH in the range of from about 5.0 to about 9.0; at least one metal-containing agent (iii), wherein said metal is selected from the group consisting of first transition series metals of oxidation state +II; and a non-ionic surfactant (iv); while ensuring that, in the final composition, the molar ratio of non-complexed calcium ions (Ca²⁺) to the Factor VII polypeptide is lower than 0.5.

59. The method according to claim 57, wherein the method comprises the step of providing the Factor VII polypeptide at a concentration of at least 0.01 mg/mL (i) in a solution comprising
a buffering agent (ii) suitable for keeping pH in the range of from about 5.0 to about 9.0; and at least one stabilising agent (iiib) comprising a —C(═N-Z¹-R¹)—NH-Z²-R² motif, wherein
Z¹ and Z² independently are selected from the group consisting of —O—, —S—, —NR^{H}— and a single bond, where RH is selected from the group consisting of hydrogen, C₁₋₄-alkyl, aryl and arylmethyl, and R¹ and R² independently are selected from the group consisting of hydrogen, optionally substituted C₁₋₆-alkyl, optionally substituted C₂₋₆-alkenyl, optionally substituted aryl, optionally substituted heterocyclyl, or
Z² and R² are as defined above and —C═N-Z¹-R¹ forms part of a heterocyclic ring, or
Z¹ and R¹ are as defined above and —C—NH-Z²-R² forms part of a heterocyclic ring, or —C(═N-Z¹-R¹)—NH-Z²-R² forms a hetercyclic ring wherein -Z¹-R¹-R²-Z²- is a biradical;
while ensuring that, in the final composition, the molar ratio of non-complexed calcium ions (Ca²⁺) to the Factor VII polypeptide is lower than 0.5.

60. A method for treating a Factor VII-responsive syndrome, the method comprising administering to a subject in need thereof an effective amount of a composition according to claim 1.

61. An air-tight, at least partially filled container containing a liquid, aqueous pharmaceutical composition as defined in claim 1, and optionally an inert gas, said container comprising (i) a wall portion and (ii) one or more closure means not constituting part of said wall portion.

62. The container according to claim 61, wherein the composition does not comprise a preservative (vii).

63. The container according to claim 61, wherein the container inner wall material is a material selected from the group consisting of silica-coated glass, silicone-coated glass, polymers of non-cyclic olefins, cycloolefin polymers, and cycloolefin/linear olefin copolymers.

64. The container according to claim 61, wherein said container is a vial or cartridge comprising a closure means which comprises a needle-penetrable, self-sealing elastomeric septum.

65. The container according to claim 61, wherein said container is a cartridge further comprising a displaceable piston means whereby liquid present in said container may be expelled from said container.
